(19) United States
(12) Patent Application Publication
(10) Pub. No.: US 2009/0320569 A1
(43) Pub. Date: Dec. 31, 2009

(54) Adjustable height liquid level management tools and systems

(76) Inventors: Keith R. Haslem, (US); Lee B. Golter, (US); Fred Burtlett, Duchesne, UT (US)

Correspondence Address: EDWIN L. HARTZ, 619 MAIN STREET, SUITE 115, GRAND JUNCTION, CO, 81501, US

(21) Appl. No.: 12/152,492

(22) Filed: May 15, 2008

Related U.S. Application Data
(60) Provisional application No. 60/930,389, filed on May 16, 2007.

(51) Int. Cl. F16H 25/20; G01N 33/00; H01L 43/06

(52) U.S. Cl. 73/61.41; 74/89.23; 324/207.2

(57) ABSTRACT

A lift mechanism positioned on a tank containing potentially explosive gases, corrosive material and/or poisonous material with the motor of the mechanism on top of the lift and more than 5 feet away from any vent or outlet of the tank and one or more devices inside the tank being moved and positioned by the lift mechanism.

### CROSS-REFERENCE TO RELATED APPLICATIONS

This application claims priority to provisional application U.S. Ser. No. 60/930,389 filed May 16, 2007, which is incorporated herein as though set forth in full.

### FIELD OF THE INVENTION

This invention relates to the management of stratified liquids in a container, and more particularly, to the management of stratified liquids in potentially explosive environments and/or corrosive and/or poisonous environments by use of variable height inlet/outlet liquid management tools, to the determination of the type of liquid and the location of the transition between liquids and to the automation of the management of stratified liquids.

### BACKGROUND OF THE INVENTION

Mixed liquids having differing specific gravities will naturally stratify while standing in a container. This occurs with many liquids in many different disciplines. For example, liquids from gas wells that are separated from the gas and stored in a tank, such as a 400-barrel or a 500-barrel tank, stratify. These liquids include hydrocarbons, water and various contaminants. The lightest (lowest specific gravity) liquid is clean oil and condensate and forms as the top layer (oil floats on water). The next layers from top down are dirty oil (a layer of dirty oil, contaminates and water), waste oil, water, and a bottom layer of sediment and water (BS&W).

Another example, is water tanks in warm climates where extra lubricant (oil) is required for pumps and the oil enters the water tank and floats on the water. The oil has to be periodically removed from the potable water in the tanks.

The invention will be described in connection with stratified liquids from gas and oil wells. However, as noted, the apparatus and methods of liquid management, both manually and by automation, are not limited to these stratified liquids. The apparatus may be designed for and may be useful in managing stratified liquids in an open container or in a closed container where the liquids include potentially explosive gases, corrosive material, and/or poisonous material. The various methods of managing liquids are useful in accessing and removing any selected layer of stratified liquids. Additionally, the apparatus and methods of managing liquids are useful in adding a material in some form to one or more of the stratified liquids. The material added may be an emulsifier or flocculent or some other material that may aid separation and stratification without mixing or contaminating neighboring layers of liquid, or may serve some other purpose.

Liquids from gas wells containing valuable light oil are present in the storage tanks at the gas wells and at the compressor stations associated with gas wells. The water removed from storage tanks at gas wells and at compressor stations is presently transported by water truck to a water plant. This produced water is initially placed in produced water tanks for ease of off-loading the trucks, for storing the water and to control the flow through a down-stream heater/separator where some of the remaining oil is separated from the other liquids (primarily water). Stratification takes place in the produced water tanks at the water plant as well as at the gas wells and compressor stations,

As noted, water is present at the output of many gas wells as part of the gas and liquid production from the wells. Water is also present when used in drilling gas wells and oil wells, as well as in forcing oil from an oil well. This water is recovered, stored in storage tanks and later used, with or without further processing. Oftentimes, hydrocarbons are present in these storage tanks and need to be removed. The removal generally includes recovery and sale of the hydrocarbons.

### SUMMARY OF THE INVENTION

Because conditions or opportunities for processing or disposing of liquids depend on various external factors such as availability of transportation trucks, disposal space, maintenance delays and changing demand or price, it is desirable to have flexibility in monitoring and removing specific liquids according to best management procedures.

Thus, it is an object of the present invention to manage the liquids in storage tanks, including, but not limited to tanks, at gas wells, compressor stations and water plants. Further, it is an object to manage liquids in water tanks and other containers of stratified liquids to reduce man hours, to reduce vehicular traffic and to improve the efficiency and safety of operation. By managing the liquids, fuller loads may be transferred to reduce vehicular traffic and pursuant to this invention, the liquids may be managed onsite and/or offsite to reduce manpower and vehicular traffic.

It is the primary object of the present invention to provide an improved method of filling and emptying storage or process tanks containing stratified liquids in a technically simple and economical manner. For this purpose a variable height inlet/outlet liquid management tool is provided. An orifice, that serves as the inlet or the outlet, is movable in a container of stratified liquids to a selected height or position for accessing and removing a selected liquid or for adding material to a selected liquid through a conduit having a first end coupled to the orifice in the tank and a second end coupled to an outlet to the exterior of the tank.

Advantages of employing a variable height orifice for managing stratified liquids are fully explained in International Application Number PCT/US2006/00479 filed Feb. 8, 2006, assigned to the same Assignee as this application, and incorporated herein in its entirety by this reference as though set forth in full.

Various apparatus or lift mechanisms for positioning the variable height orifice are shown and described in this PCT application. One apparatus shown (FIGS. 91-99) and described employs a lead screw and nut inside the container of liquids. Some liquids are highly caustic and/or corrosive and, therefore, it is desirable to keep the lead screw and nut out of these liquids. Thus, in accordance with this invention, the lift mechanism (postioner) is placed outside the tank and is not contacted by the liquids in the tank. Additionally, for use in certain fields, such as gas fields, the higher energy devices, including motors, may be placed at a sufficient distance from the container that more costly explosion proof devices are not required.

A particular and significant advantage of managing liquids by the tools and system of this invention is where the liquids are in a highly flammable or explosive environment. The tools provide a safe means of choosing which liquid to add to or extract in this type of environment. The tools do not generate static or sparks and are grounded for any transient static charge that may originate from an operator's clothing. In addition, the tools or apparatus attached to a container or tank maintain a seal that prevents fluids, which may be explosive or poisonous, from escaping into the atmosphere.

The tool includes a lift mechanism for positioning a variable height inlet/outlet orifice and/or a type of liquid discrimination sensor. Advantageously, the higher electrical energy components may be more than 5 feet away from any vent or outlet of the container on which the lift is used. All other components are low energy and thus are intrinsically safe. The area within 5 feet of a vent or outlet is classified in the gas industry as Class 1 Div. 2 and the motor and other high energy components of the lift are best placed outside this area.

The lift consists of an elongated housing which may be square or rectangular in cross-section or some other suitable configuration. Particularly useful dimensions for square and rectangular cross-sections are 3″ by 3″, 4″ by 4″, 5″ by 5″ and 3″ by 4″ for the gas and oil fields. Other dimensions may be useful in these fields and other fields.

The housing may be slightly more than 5 feet in length to place the higher energy components beyond the 5-foot classification environment. A lead screw and lead screw nut are located in the housing with the lead screw nut moving vertically upon rotation of the lead screw. The lead screw is suspended from and turns in thrust bearings mounted at the top of the housing.

Attached to the bottom of the lead screw nut is a hollow tube of sufficient length to extend into a container upon which the lift is mounted. The lead screw nut is preferably metal, such as bronze, for durability and also to provide electrical contact for grounding and avoiding static electricity. The lead screw nut may also be plastic, such as ultrahigh molecular weight plastic, with carbon filling to provide electrical contact. In either case, a metal plate is attached to the bottom of the nut for attachment of the hollow tube of the lift mechanism.

A variable height inlet/outlet orifice and/or liquid discrimination sensor is carried by the hollow tube to be selectively positioned inside the container. The hollow tube passes through a housing that is attached to the top of the container by means such as mating threads. The housing contains bearings for movement of the hollow tube inside the housing and to ground the housing to the container. The hollow tube is also grounded in this way. The housing further contains seals to prevent vapors from the container escaping to the outside of the container through the lift.

The housing is made with the smallest cross-section possible for the particular use of the lift mechanism. The small size is to present the smallest silhouette to the elements and, particularly, to wind on top of the container. A motor is mounted on top of the lift mechanism and is coupled to the lead screw to control the operation of the lead screw. A love-joy coupling is provided at the top of the lead screw for easy coupling to the motor shaft. The position of the lead screw nut and the thus the position of the orifice and/or sensor, inside the container, is monitored and determined by Hall effect sensors and a small magnet carried by the lead screw nut. At least two Hall effect sensors are mounted in the side of the housing, on the side of the lead screw nut where the magnet is located. One sensor is positioned near the top of the housing where the lead screw nut is to stop in its upward movement and the second sensor is positioned near the bottom of the housing where the lead screw nut is to stop on the downward stroke. For tall housings, additional sensors may be placed in the wall of the housing to sense the magnet as the lead screw nut passes by the sensor to provide a faster indication of the location of the lead screw nut. In cooperation with the Hall effect sensors, an encoder is mounted on the shaft of the motor with a magnet embedded in the encoder, which may advantageously include a rotating disk and sensor. A Hall effect sensor is attached to the housing or support for the motor in magnetic field contact with the magnet so that as the motor shaft rotates, the direction and speed of rotation are sensed by the Hall effect sensor. The lead screw may have 6 threads per inch so that for each revolution, the lead screw nut and hollow tube moves ⅙th of an inch. Thus, by counting the number of revolutions, the distance traveled by the lead nut and hollow tube is attained.

The position of the lead screw and, thus, the position of the device, component or instrument in the container may be sensed by other devices such as magnetostrictive sensors, infrared sensors, and laser distance sensors, for example. The management of stratified liquids may be automated for on-site control or off-site control. The level information and the position information may be accessed by use of a man machine interface having at least a readout; and preferably a display of the information and a memory for recording the data. A pumper or operator may access the information from an interface mounted in a truck, mounted on the container, mounted in an instrument box or one that is hand-held.

The operation of the variable height inlet/outlet orifice and/or sensor in a container and the management of the liquids may be fully automated and controlled at a central station using the SCADA (Supervisory Control and Data Acquisition) approach. For off-site management of liquids in one or more containers at a plurality of sites, such as well sites, compressor stations and water plants, there is provided a remote terminal unit at each site. Each container being managed at a site has a level sensor and a motor controlling the position of the orifice in the container. At the output, there is at least one automated valve between the outlet of the container and a storage container and/or a transport vehicle.

Objects, features and advantages of this invention will become apparent from a consideration of the above, the following description, the appended claims and the accompanying drawings.

### BRIEF DESCRIPTION OF THE DRAWINGS

FIGS. 1A and 1B, joined together, are an elevation view, partially in cross-section, of the lift mechanism, in accordance with this invention;

FIG. 2 is a schematic-elevation view illustrating the position of the stabilizing plates in use, in accordance with this invention;

FIG. 3 is a cross-sectional view of a portion of FIG. 2 along section lines 3-3, in accordance with this invention;

FIG. 4 is a cross-sectional view along the section lines 4-4 of FIG. 2, in accordance with this invention;

FIG. 5 is a cross-sectional view along the section lines 5-5 in FIG. 2, in accordance with this invention;

FIG. 6 is an elevation view, partially in cross-section, of various components inside a container, positioned by the lift mechanism, in accordance with this invention;

FIG. 7 is a block and schematic diagram of the electrical wiring and control elements, in accordance with this invention; and

FIG. 8 is an elevation view with the housing partially broken away to show an alternative position sensor, in accordance with this invention.

### DESCRIPTION OF PREFERRED EMBODIMENTS

The positioner or lift mechanism of this invention may be used to move many different devices, components or instruments inside a container. One particularly advantageous use is where the device, component or instrument is in a container of corrosive liquids. The drive portion of the lift mechanism is outside the tank and does not contact the corrosive liquid.

Another advantageous use of the lift mechanism is where the container holds liquids, which include potentially explosive gases. In the gas field industry, there are certain classifications of volatility inside such a container and in the areas outside the container. In particular, the area that is within 5 feet of an opening or vent of the container is classified as Class 1 Div. 2 and all mechanical or electrical devices inside this 5-foot area have to be explosion proof. To avoid the expense of explosion proof motors, the lift mechanism of this invention may have a housing of sufficient length to place the motor and its controls outside the 5-foot area so that the motor and its controls do not have to be explosion proof.

Other components of the lift mechanism that are within the 5-foot area immediately outside the container need to be of sufficiently low current and voltage to be intrinsically safe in this environment. The lift mechanism of this invention employs such devices inside the 5-foot area to be intrinsically safe when the lift mechanism is used on a container which holds explosive liquids.

The lift mechanism 1 consists of a housing 10 having a selected cross-section and length. When the lift mechanism is employed on top of storage tanks found in gas fields, it is desirable that the lift mechanism be as short as possible. However, because of the 5-foot classification area around storage tanks in gas fields, it is desirable that the housing 10 be at least 5 feet in length so that the motor that is used to drive the lift mechanism is outside the Class 1 Div. 2 area.

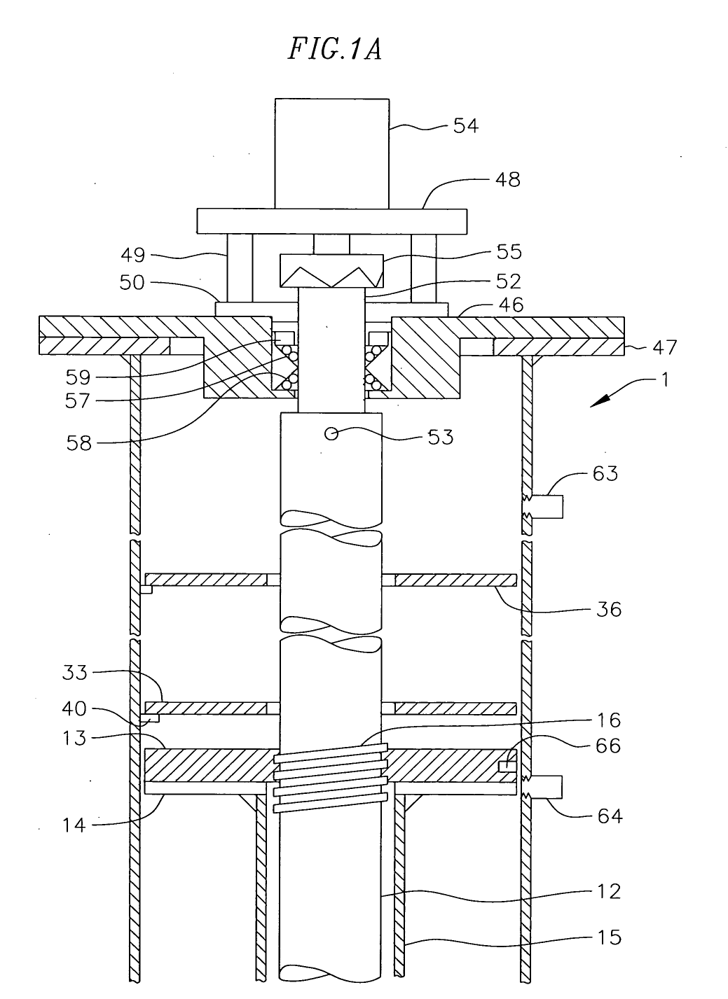

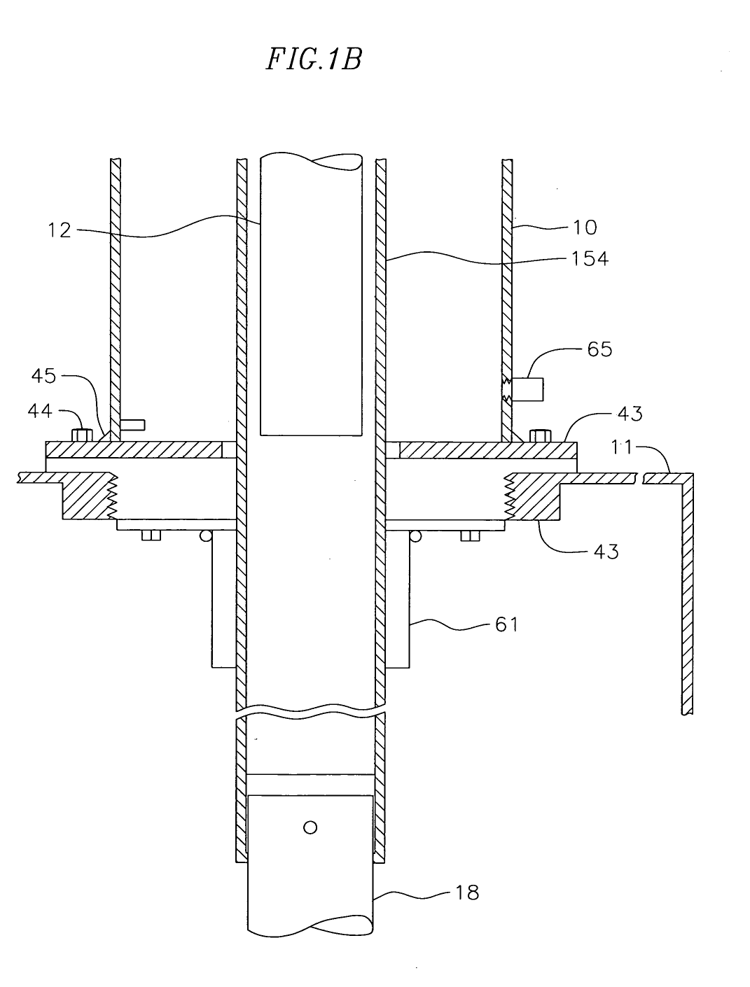

The lift mechanism further consists of a lead screw 12 that is contained in the housing 10 and rotates within the housing 10. The lead screw 12 is threaded over its entire length; however, threads 16 are only shown in the area of a lead nut 13 on the drawing of FIG. 1. A lead nut 13 is positioned inside the housing 10 on the lead screw 12 and moves vertically as the lead screw 12 rotates. The lead screw nut 13 is square in cross-section and is slightly smaller than the internal dimensions of the housing. Thus, the lead screw nut 13 is kept from turning with the lead screw and only moves vertically in the housing 10.

The nut 13 may advantageously be made of ultra-high molecular weight plastic with carbon fibers to provide electrical contact. The nut 13 may also be made of metal, such as brass, for durability and reliability.

The lead screw may have selected threads per inch to correspond to the use of the lift mechanism. For positioning orifices and to move liquid discrimination sensors, six threads per inch is satisfactory. Thus for every full rotation of the lead screw and motor shaft, if driven by a motor, the lead screw nut 13 moves vertically ⅙th of an inch.

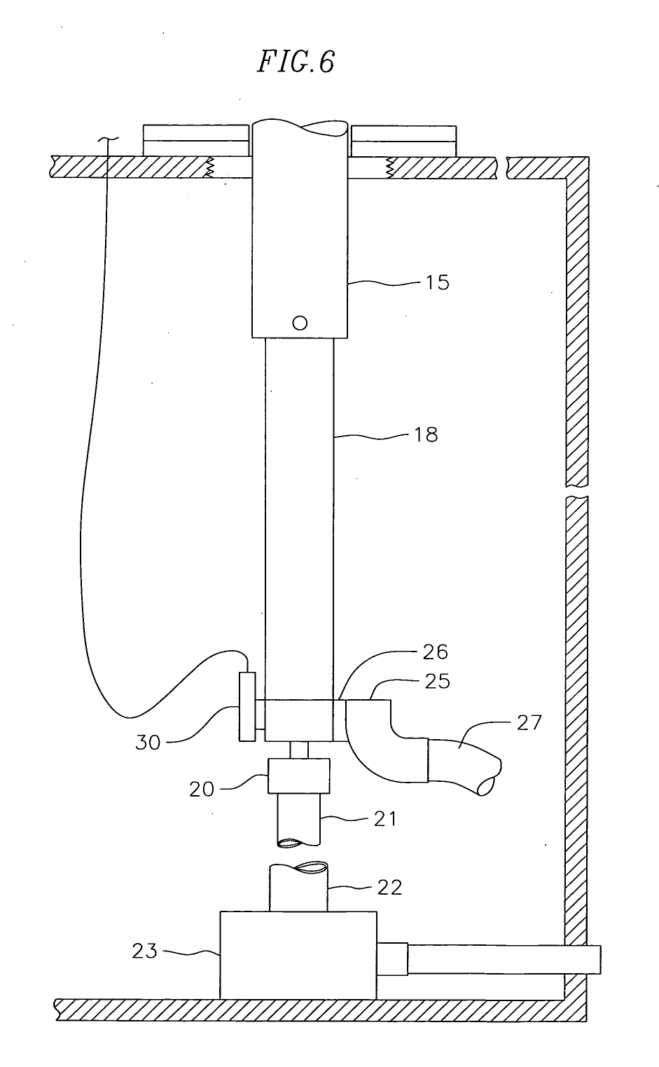

A square metal plate 14 is attached to the bottom of the lead nut 13 and moves with the lead nut 13. A tubular rod 15 is attached to metal plate 14 by some means, such as welding for example, and thus moves with the metal plate 14 and lead nut 13. Tubular rod 15 extends down into the container 11 for attachment to a rod inside the tank to position the device, component or instrument that is to be positioned inside the container. The coupling of the tubular rod 15 to a carrier rod 18, inside the container 11, is shown in FIG. 1B and FIG. 6.

Two types of variable height orifice mechanisms that are fully described in the above-identified PCT Application are schematically illustrated in FIG. 6. One variable height orifice 20 is associated with rigid telescoping pipe 21 and 22 and a standpipe 23. A second variable height orifice 25 is carried by a housing 26 attached to the carrier rod 18. The second variable height orifice 25 is coupled to the input end of a conduit, or flexible hose 27. Either variable height orifice 20 or 25 may be positioned by the lift mechanism 1 of FIG. 1.

In conjunction with one of the variable height orifices, or independently of the orifices, a discrimination sensor 30 may be carried and positioned by the carrier rod 18 and carrier 26. The discrimination sensor 30 may be one of the sensors described in U.S. patent application Ser. No. 11/413,774 filed Apr. 28, 2006, U.S. Provisional Patent Application No. 60/810,013 filed May 31, 2006 (PCT/US2007/012,681 filed May 30, 2007) and/or U.S. Provisional Patent Application No. 60/836,762 filed Aug. 10, 2006 (U.S. Ser. No. 11/891,283 filed Aug. 9, 2007). All of these applications are assigned to the same assignee as this application and the disclosure in each of these applications is incorporated herein in its entirety by this reference as though set forth in full.

Depending upon the length of the housing 10 and the lead screw 12, one or more stabilizing plates 32 and 33 may be employed. The plates 32 and 33 rest on top of the lead nut 13 when it is in its uppermost position. As the lead nut 13 moves vertically downward, the stabilizing plates 32 and 33 come to rest at selected positions inside the housing 10. For a 5-foot long housing 10 and a ⅝-inch diameter lead screw 12, two stabilizing plates are generally sufficient. In this case, the stabilizing plate 32 comes to rest at about 1½ feet down from the top and stabilizing plate 33 comes to rest at about 3 feet down from the top. As the lead nut 13 continues on toward the bottom, the stabilizing plates 32 and 33 are designed to eliminate or minimize bending or flexing of the lead screw 12.

Three pins extend into the housing 10 at selected elevations inside the housing. The pins have varying lengths that correspond to varying depths of slots in the stabilizing plates 32 and 33 and the lead nut 13. Relatively short pins 36 are positioned at the upper point to catch the first stabilizing plate 32. The stabilizing plate 33 and lead nut 13 have indentations 38 and 39, respectively, to pass by the short pins 36. Medium length pins 40 stop the second stabilizing plate 33 at the selected height of the pins 40. The indentations 39 in lead nut 13 are deep enough to permit lead nut 13 to pass by, or below, the pins 40. Additional pins 41 may be provided near the bottom of the housing 10 to prevent the lead nut from passing below the pins 41.

A threaded bearing and seal housing 42 is threaded into a threaded coupling 43 at the top of the container 11. The bearings and seals in the housing 42 that contact the tubular rod 15 are not shown in FIG. 1 but may be similar to the bearings and seals shown in FIGS. 8, 10 and 11 of the above-identified PCT Application /US2006/004479 (U.S. Ser. No. 11/884,100 filed Aug. 8, 2007). The bearings and seals in housing 42 provide electrical contact with the tubular rod 15 for grounding of the components of the lift mechanism and to avoid sparking and static electricity. Also, at least some of the seals may be wiper seals to clean the rod 15 as it moves back up into the housing 10.

The housing 10 has a bottom mounting plate 43 that is attached to the bearing and seal housing 42 by some means such as bolts 44. The square housing 10 is attached to the bottom mounting plate 43 by some means such as welding 45. The lead screw 12 is mounted and held in place in a bearing and seal housing 46 mounted at the top of the housing 10 on a circular flange 47.

A motor-mounting plate 48 extends above the bearing and seal housing 46 by a cylindrical extension 49. The cylindrical extension 49 couples the motor mounting plate 48 to a circular mounting plate 50 that is attached to the bearing and seal housing 46 by some means, such as bolts (not shown). The lead screw 12 is coupled to an extension rod 52 that is of smaller diameter than the lead screw 12. The extension rod 52 extends into a hole drilled into the top of lead screw 12 and is coupled to lead screw 12 by a pin 53.

A gear mechanism and/or motor 54 is attached to the mounting plate 48. A typical coupling between a motor 54 and shaft 52 is a Love-joy coupling 55 as shown in FIG. 1.

Lead screw 12 is supported from the bearing and shaft housing 46 by a pair of thrust bearings 57 and 58. The thrust bearings 57 and 58 are held in place and under proper tension by thrust nut 59.

An extension or nozzle 61 is provided at the lower-end of the lift mechanism 1 to give added stability to the tubular rod 15 as it extends into the tank or container 11.

The controls for the lift mechanism 1 include a position-sensor system. The position-sensor system of FIG. 1 consists of one or more Hall effect sensors 63, 64 and 65 threaded into the side of housing 10. A small magnet 66 is carried by the lead nut 13 in vertical alignment with the sensors 63-65. As the magnet 66, on lead nut 13, passes a sensor, the sensor is turned on and off to indicate the position of the lead nut 13. Sensor 63 is positioned near the top of the housing 10 to act as a limit switch beyond which the lead nut 13 may not pass. As a limit-switch, sensor 63 may shut off the power to the motor 54 to stop the rotation of the lead screw 12 and the movement of the lead nut 13. Similarly, sensor 65 is positioned near the bottom of the housing 10 to limit the downward movement of the lead nut 13. As sensor 65 detects the magnet 64 in the lead nut 13, it may also shut off the power to the motor 54 to stop the movement of the lead nut 13.

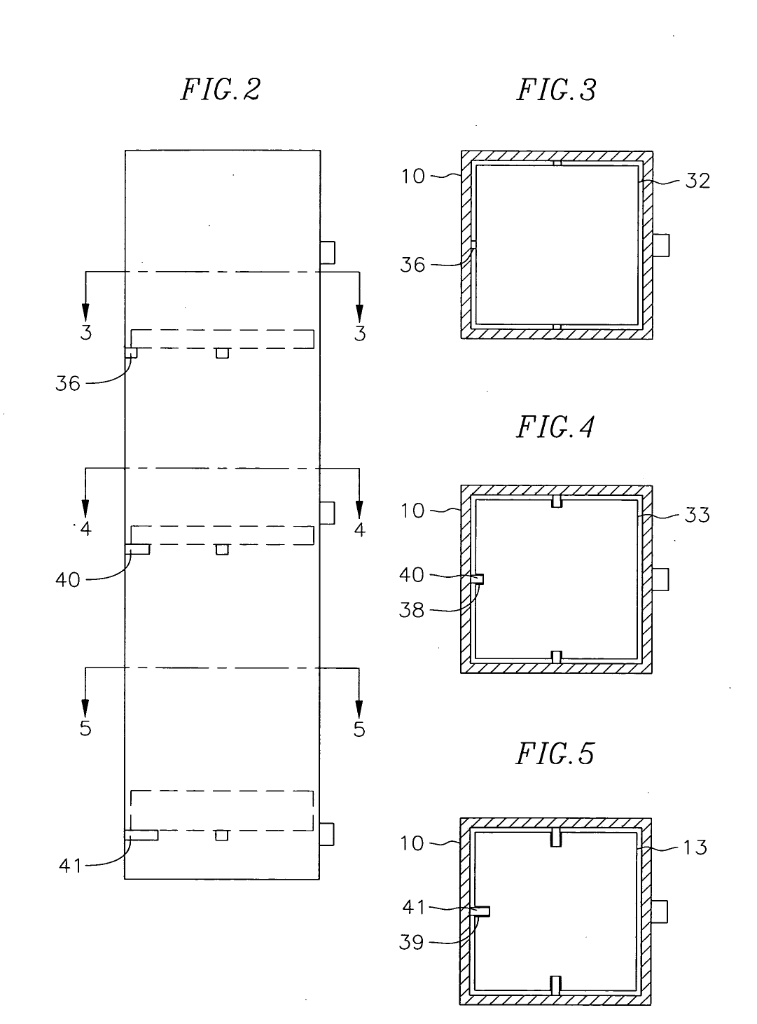

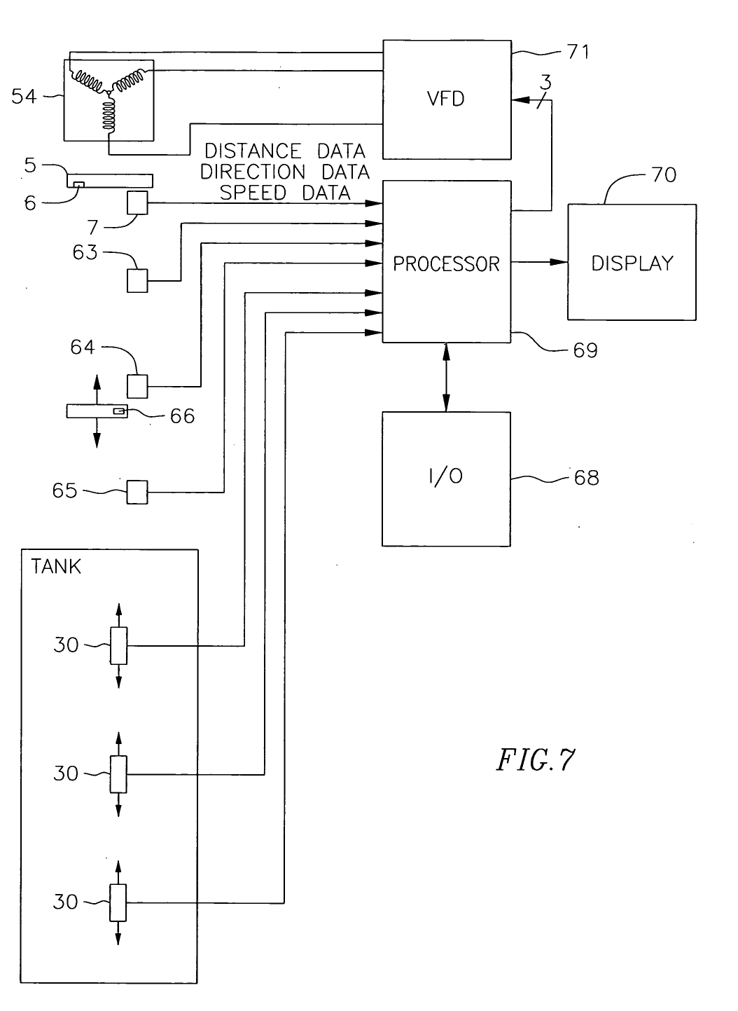

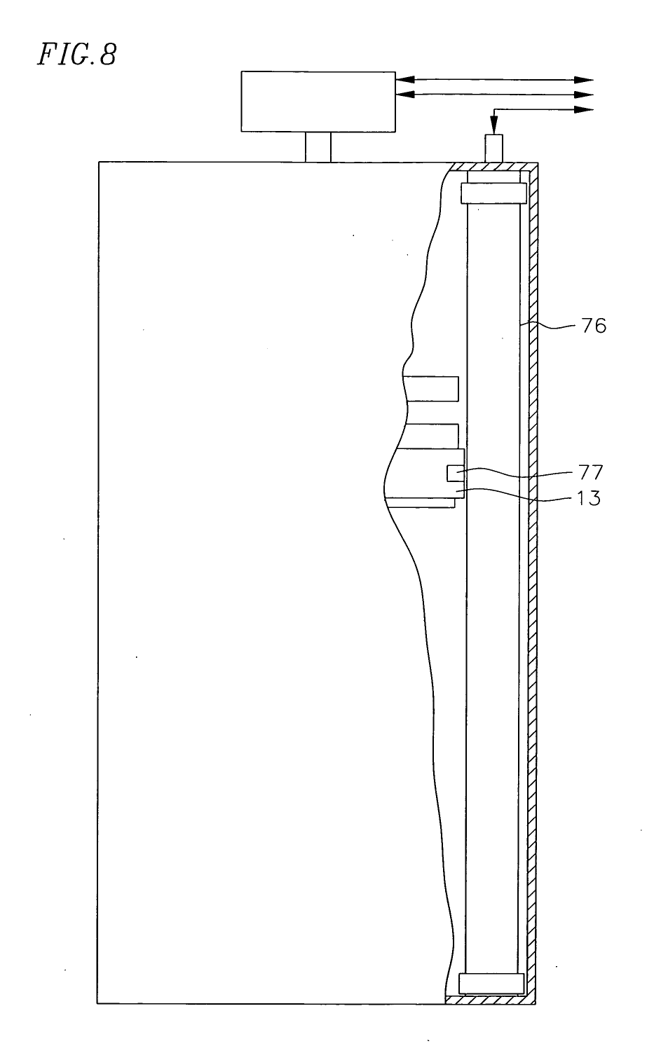

The operation of the lift mechanism can be better understood by reference to FIG. 7 in connection with the devices shown in FIGS. 1-6. The operation will be described with one or more discrimination sensors 30 inside the tank. Variable height orifices 20 and/or 25 may also be inside the tank. The position of those orifices may be controlled based on the output of the discrimination sensor 30. In FIG. 7 a plurality of sensors 30 are depicted, as would be the case if the tank or container 11 is so tall that the travel of the tubular rod 15 cannot cover the full height of the tank 11. For example, it is common in the gas fields for the storage tanks to be 20-feet tall and it is typically not desired to have a lift mechanism on top of the tank be 20-feet tall. The lift mechanism 10 is as short as possible (but greater than 5 feet in length for some uses). Additionally, the lift has a silhouette as small as possible to limit the bending stresses on the lift mechanism from winds where the lift mechanism is employed. With a housing 10 that is slightly longer than 5 feet, the travel of the tubular rod 15 inside the tank 11 may be 5 feet. If the height of the liquid in the tank 11 is 15 feet, then 3 sensors 30, spaced 5 feet apart and traveling vertically in the tank 11, may cover the full height of the liquid to provide a discrimination output to indicate the type of liquid in the tank and the transition level between the types of liquid. For example, liquids from a gas well will stratify with clean oil on top, followed by dirty oil, waste oil, water and bottom sediment and water. The sensors 30 will detect the type of liquid and the transition from clean oil to dirty oil, dirty oil to waste oil, waste oil to water and water to bottom sediment and water. The operation of the discrimination sensors and, particularly, the preferred type is disclosed in the above-referenced U.S. Provisional Application No. 60/836,762 filed Aug. 10, 2006 (U.S. Ser. No. 11/891,283 filed Aug. 9, 2007).

The operation of the lift mechanism and the position of orifices or the travel and position of discrimination sensors may be controlled by an input-output device 68. Input/output device 68 may have a keyboard or touch screen for controlling the operation of the system. The input/output device 68 is the human machine interface for the system. The operation of the system is controlled by a processor 69 that has inputs from all of the sensors and the input/output device 68. A display 70 may display the contents of the tank 11 either graphically or numerically. Further, the display 70 may show where the orifice is inside the tank relative to the liquids sensed by one or more of the discrimination sensors 30. The direction of travel of the tubular rod 15, the speed of travel and the distance of travel is controlled by the motor 54 in response to the processor 69 through a variable frequency device 71. The revolutions of the lead screw 12 and thus the travel of the lead nut 13 and the tubular rod 15 may be determined by sensing the revolutions of the lead screw 12 through a disk 5 mounted on the shaft extending from the motor 54. The disk 5 has a magnet 6 embedded therein which passes by a Hall effect sensor 7. The output of the Hall effect sensor 7 indicates the speed of travel and the distance of travel by the number of revolutions of the lead screw 12. The direction of travel of the lead screw nut 13 is defected by the sensors 63, 64 and 65.

There are various other devices that may be employed in place of the magnet 66 and Hall effect sensors 63, 64 and 65. For example, as shown in FIG. 8, an alternative sensor for monitoring the position of the tubular rod 15 and thus any device inside a tank, consists of a micro-pulse transducer 76 and floating magnet 77 carried by the lead nut 13. Such a sensor is available from Balluff GMBH Schurwaldstrasse 973765 Neuhausen A.D.F., Germany with a Model Number Micro-Pulse AT Transducer. Other sensors may be used such as time of flight sensors employing lasers. There are other sensors for measuring travel by devices such as lead nuts used in the lift mechanism 10 of this invention.

It is to be understood that the above-referenced arrangements are only illustrative of the application of the principles of the present invention in one or more particular applications. Numerous modifications and alternative arrangements in form, usage and details of implementation can be devised without the exercise of inventive faculty, and without departing from the principles, concepts and scope of the invention as disclosed herein. Accordingly, it is not intended that the invention be limited, but rather the scope of the invention is to be determined as claimed.

What is claimed is:

1. Apparatus for positioning one or more devices in a tank containing potentially explosive gases, corrosive material and/or poisonous material comprising a lift mechanism positioned outside the tank and a motor, as part of the lift mechanism, positioned at least 5 feet away from any vent or outlet of the tank.

2. The lift mechanism in accordance with claim 1 further comprising a housing supporting the motor, a lead screw supported in the housing and extending vertically in the housing, means for coupling the top of the lead screw to the motor, a lead nut positioned to travel vertically in the housing when the lead screw turns, a hollow tube coupled to the bottom of the lead screw nut and extending into the tank to carry one or more devices inside the tank.

3. The lift mechanism in accordance with claim 2 further comprising means for determining the position of each device inside the tank.

4. The lift mechanism in accordance with claim 3 wherein the determining means are a series of hall effect sensors positioned vertically in the side of the housing and a magnet carried by the lead screw in position to be sensed by a hall effect sensor as the lead screw nut passes in close proximity to the sensor.

5. The lift mechanism in accordance with claim 3 wherein the determining means is a disc which rotates with the shaft of the motor, one or more magnets carried by the disc and a hall effect sensor positioned to sense the passage of each magnet on the disc.

6. Apparatus for determining the type of liquid in a tank and the transition level of the transition from one type to another type comprising a lift mechanism positioned on top of the tank and having an extension into the tank to carry and position discrimination sensors and discrimination sensors vertically positioned in the tank and spaced apart a selected distance.

7. Apparatus in accordance with claim 6 wherein the distance is dependent upon the travel of the extension of the lift mechanism.
